(19) United States
(12) Patent Application Publication
(10) Pub. No.: US 2007/0162100 A1
(43) Pub. Date: Jul. 12, 2007

(54) System and method for loading implanter with prosthesis

(76) Inventor: Shlomo Gabbay, New York, NY (US)

Correspondence Address: TAROLLI, SUNDHEIM, COVELL & TUMMINO L.L.P., 1300 EAST NINTH STREET, SUITE 1700, CLEVEVLAND, OH, 44114, US

(21) Appl. No.: 11/328,546

(22) Filed: Jan. 10, 2006

(51) Int. Cl. A61F 2/06

(52) U.S. Cl. 623/1.11

(57) ABSTRACT

A system includes a prosthesis receiving portion having an opening at a first end that is spaced axially apart from a second end by a sidewall portion. The sidewall portion has a substantially smooth, radially inner sidewall that tapers from a first diameter at the opening to a second diameter at the second end of the prosthesis receiving portion. The second diameter is less than the first diameter and defines an exit aperture of the prosthesis receiving portion. A receptacle extends from the second end of the prosthesis receiving portion. The receptacle is dimensioned and configured for coaxially receiving a hollow portion of the implanter at a location relative to the exit aperture that provides a fluid transition between from the exit aperture of the prosthesis receiving portion to the receptacle, whereby loading the prosthesis from the within the prosthesis receiving portion into the hollow portion of the implanter is facilitated.

### BACKGROUND

Various types of implantable cardiovascular prostheses have been developed and corresponding approaches are utilized to implant prostheses in both human and non-human patients. For example, it is known to utilize annuloplasty rings, stents other implantable cardiac prosthetic devices for helping improve functionality of a patient's heart valve. Other types of valves (e.g., venous valves) and stents can be utilized to improve circulation in veins and other blood vessels.

In severe cases of valvular defect and/or deficiency, implantable heart valve prostheses, such as natural tissue valves, mechanical valves and biomechanical valves are employed to replace a defective valve. In most cases, to surgically implant these and other cardiac prostheses into a patient's heart, the patient typically is placed on cardiopulmonary bypass during a complicated, but common, open chest and, usually, open-heart procedure. In an effort to reduce risk to the patient, minimally-invasive implantation techniques for various cardiac prostheses are continually being developed and improved. Most of such research and development has focused on the prostheses and the devices being used to implant such devices.

There exists a need for improved systems and methods for loading the cardiovascular prostheses into implantation devices.

### SUMMARY

The present invention relates generally to a system and method for loading an implantable device into an implanter.

One aspect of the present invention provides a system for loading an implantable prosthesis into an implanter. The system includes a prosthesis receiving portion having an opening at a first end that is spaced axially apart from a second end by a sidewall portion. The sidewall portion has a substantially smooth, radially inner sidewall that tapers from a first diameter at the opening to a second diameter at the second end of the prosthesis receiving portion. The second diameter is less than the first diameter and defines an exit aperture of the prosthesis receiving portion. A receptacle extends from the second end of the prosthesis receiving portion. The receptacle is dimensioned and configured for coaxially receiving a hollow portion of the implanter at a location relative to the exit aperture that provides a fluid transition between from the exit aperture of the prosthesis receiving portion to the receptacle, whereby loading the prosthesis from the within the prosthesis receiving portion into the hollow portion of the implanter is facilitated.

Another aspect of the present invention provides a system for preparing a prosthesis for in vivo implantation. The system includes a guide member comprising: a prosthesis receiving portion having a substantially smooth, radially inner sidewall having a conical frustum cross sectional configuration that tapers from the from a first diameter at an opening at a first end of the guide member to a smaller second diameter at the second location within the guide member that is spaced apart from the first end. The guide member also includes a receptacle located adjacent the second end of the sidewall portion. An implanter has an elongated barrel having a lumen configured for receiving the prosthesis in a reduced cross-sectional dimension. The receptacle is dimensioned and configured for aligning an opening of the barrel with the second location within the guide member, whereby loading the prosthesis from the within the guide into the portion of the implanter is facilitated.

Yet another aspect of the present invention provides a method of using a system to load a prosthesis into the barrel of the implanter. The method includes inserting the barrel of the implanter within the aperture so that the opening of the barrel is adjacent and aligned with the exit aperture of the prosthesis receiving portion. A deformable prosthesis is positioned at the opening of the prosthesis receiving portion and the prosthesis is urged axially into the prosthesis receiving portion such that the inner sidewall of the prosthesis receiving portion engages the exterior of the prosthesis and causes a cross-sectional dimension of the prosthesis to reduce commensurate with the cross sectional dimension of the inner sidewall being engaged by the prosthesis. The prosthesis is pushed through the exit aperture and through the opening of the barrel such that at least a portion of the prosthesis resides within the barrel of the implanter.

### BRIEF DESCRIPTION OF THE DRAWINGS

FIG. 1 depicts an example of a loading system that can be utilized to load a prosthesis into an implanter according to an aspect of the present invention.

FIG. 2 is a partial cross sectional view taken along line 2-2 in FIG, 1.

FIG. 3 depicts a first portion of a procedure for loading a prosthesis into an implanter using the loading system of FIG. 1 according to an aspect of the present invention.

FIG. 4 depicts a second portion of a procedure for loading a prosthesis into an implanter using the loading system of FIG. 1 according to an aspect of the present invention.

FIG. 5 depicts a third portion of a procedure for loading a prosthesis into an implanter using the loading system of FIG. I according to an aspect of the present invention.

FIG. 6 depicts a first portion of a procedure for loading a prosthesis into an implanter using a loading system according to another aspect of the present invention.

FIG. 6A depicts a front view of the pusher member in a first condition taken along line 6A-6A in FIG. 6.

FIG. 7 depicts a second portion of the procedure of FIG. 6 according to an aspect of the present invention.

FIG. 7A depicts a sectional view taken along line 7A-7A in FIG. 7, illustrating the pusher member in a second condition.

### DETAILED DESCRIPTION

In the area of minimally invasive cardiovascular surgery, several types of prostheses, including heart valves, venous valves, stents, annuloplasty rings and other apparatuses, can be compressed to a smaller diameter to facilitate their positioning to a desired implantation site (e.g., within a patient's heart). For instance, many such devices may have a substantially C-shaped or substantially cylindrical configuration when in an expanded state, as intended for replacing or augmenting operation of anatomical features, such as a heart valve. Some of the prostheses intended for minimally invasive surgical implantation include spikes, barbs or other protrusions that extend outwardly from the prosthesis. Accordingly, when handling the prosthesis, traditional sterile gloves can rip or be punctured by the spikes or barbs. The present invention provides a system and method for reducing the cross-sectional dimension (e.g., diameter) of a prosthesis to facilitate loading the prosthesis into an implanter.

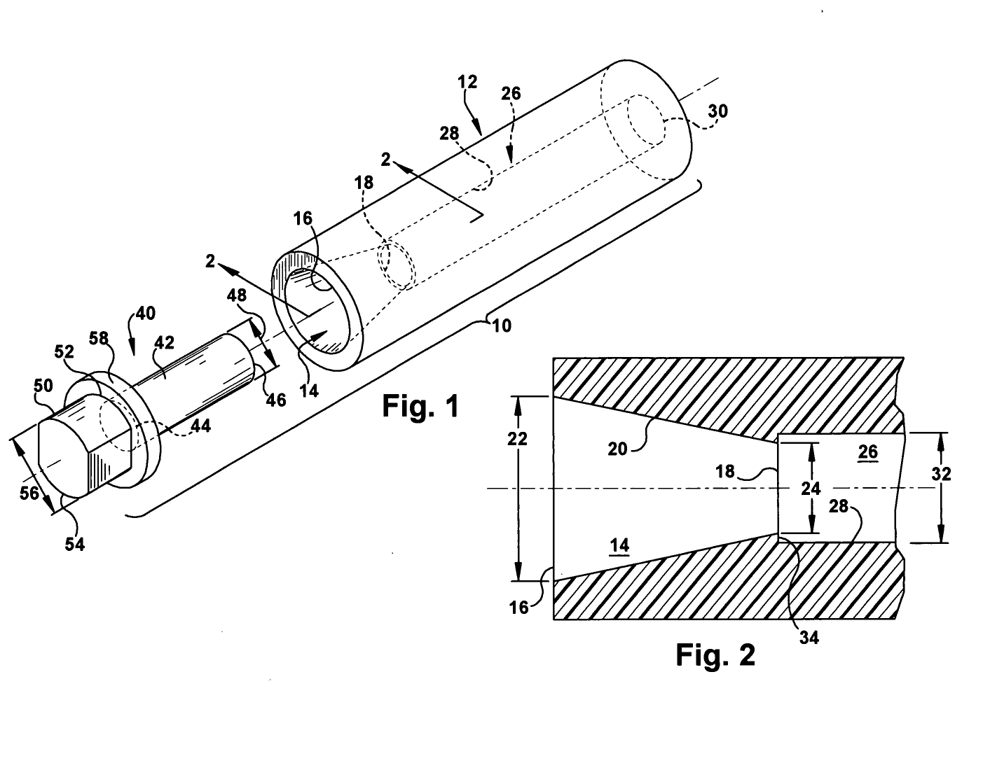

FIGS. 1 and 2 depict an example of a system 10 that can be utilized to load a prosthesis into an implanter according to an aspect of the present invention. The system 10 includes a guide member 12 having a prosthesis receiving portion 14. The prosthesis receiving portion 14 includes an opening 16 at a first end that is spaced apart from an exit aperture 18 by a substantially conical interior sidewall portion 20. The opening has a diameter 22 that is greater than the diameter 24 of the exit aperture, with the sidewall tapering from the larger diameter at the opening 16 to the smaller diameter at the exit aperture 18. The particular dimensions of the diameters 22 and 24 can be configured according to the starting (or expanded) size of prosthesis and the desired ending (or reduced cross-sectional) size of the prosthesis. As described herein, the desired ending size will be defined by the inner diameter of the implanter barrel in which the prosthesis is being loaded.

In the example of FIGS. 1 and 2, the interior sidewall portion 20 is illustrated as having a conical frustum (or frusto-conical) cross sectional configuration that extends between the opening 16 and the exit aperture 18 of the prosthesis receiving portion 14. For example, the interior sidewall portion 20 may be configured with an angle that is less than approximately 45 degrees, such as in a range from about 10 degrees to about 20 degrees, relative to a central longitudinal axis extending through the guide member 12. It is to be understood that the shape of the prosthesis receiving portion 14 is not limited to the shape of a conical frustum. For example, other shapes or combinations of shapes, including one or more curved portions, can be used to provide the interior sidewall 20 of the prosthesis receiving portion 14 according to an aspect of the present invention.

The guide member 12 also includes a receptacle 26 extending from the second end 18 of the prosthesis receiving portion 14. The receptacle 26 is dimensioned and configured for axially aligning an opening of the implanter (e.g., a barrel or other generally tubular structure in which the prosthesis is being loaded) with the exit aperture 18 of the prosthesis receiving portion 14. The particular configuration of the receptacle 26 can vary from that shown in FIGS. 1 and 2. For instance, the receptacle 26 can be substantially shorter and/or include one or more fasteners or clips that help hold the barrel of the implanter substantially in alignment with the exit aperture 18, such that the prosthesis can be easily transferred from the prosthesis receiving portion 14 into the barrel.

In the example of FIGS. 1 and 2, the receptacle 26 is depicted as including a lumen 28 that extends longitudinally from the exit aperture 18 and terminates in an implanter receiving opening 30. The lumen 28 is defined by an interior sidewall of the receptacle 26 that extends between the exit aperture 18 and the implanter receiving opening 30. The lumen 28 has a diameter 32 (at least near the prosthesis receiving portion 14) that is at least the same length as the diameter 24 of the exit aperture 18. As one example, the diameter 32 of the lumen 28 is slightly greater (e.g., by about 2 mm to about 5 mm greater) than the diameter 24 of the exit aperture 18. By providing the lumen 28 with a greater diameter than the exit aperture 18, while also being substantially coaxial, a substantially annular shoulder 34 is provided at the juncture of the sidewall 20 and the lumen 28. The shoulder 34 thus may inhibit axial insertion of a barrel of an implanter into the receptacle 26 beyond the shoulder while still providing for fluid communication from the prosthesis receiving portion 14 into the receptacle.

As an example, the shoulder 34 extends substantially radially from the exit aperture to the sidewall that defines the lumen 28. The shoulder 34 provides a stop that inhibits insertion of the implanter beyond the shoulder. That is, the barrel of the implanter (having an outer diameter that approximates the diameter 32 of the lumen) can be inserted into the lumen 26 such that the opening of the barrel engages the shoulder 34 while the barrel is substantially axially aligned with the exit aperture 18. In this way, the shoulder 34 provides a desired transition between the prosthesis receiving portion 14 that facilitates loading the prosthesis into the implanter barrel.

The prosthesis receiving portion 14 and the receptacle 26 can be formed as a monolithic structure. By monolithic structure, it is meant that the receptacle 26 and the prosthesis receiving portion 14 are integrally formed as a single piece; although, it does not require that the structure include only one type of material. The guide member can be formed of one or more materials. Those skilled in the art will understand and appreciate various manufacturing techniques that can be employed to make the guide member 12, including injection molding, stamping, casting, extrusion, machining, to name a few, or any combination thereof The guide member 10 is not limited to any of method of manufacture, however.

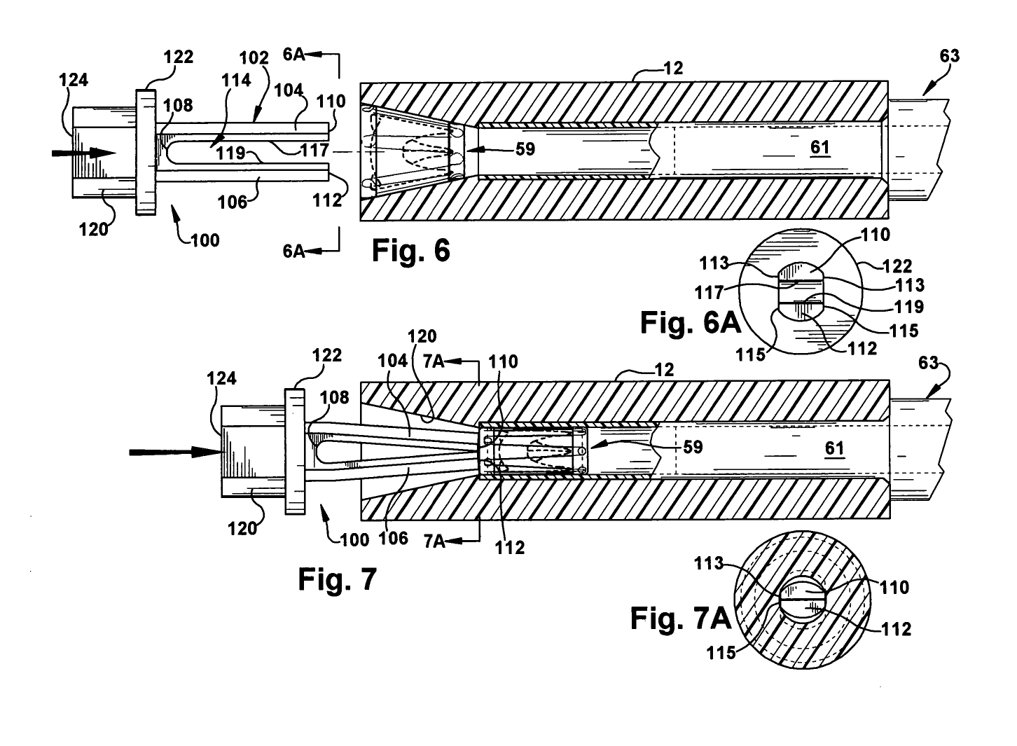

As depicted in FIG. 1, the system 10 can also include a pusher member 40. The pusher member 40 includes at least one elongated rod 42 that extends axially from a first end 44 and terminates in a second end 46. The second end 46 of the rod 42 can be substantially flat (e.g., substantially planar) or otherwise configured for engaging an end of the prosthesis. The diameter 48 of the rod 42 may be fixed along its length. The diameter 48 of the rod 42 at the end 48 can be between the diameter 22 of the opening 16 and the diameter 24 of the exit aperture 18. For example, by dimensioning the diameter 48 of the rod 42 to approximate the diameter of the exit aperture 18 and providing the elongated rod 42 with an axial length that is at least equal to or greater than the axial length of the prosthesis receiving portion 14, the rod can be inserted completely into the prosthesis receiving portion 14. Alternatively, the rod 42 can be configured as including two (or more) spaced apart elongated members configured to provide a variable diameter. For example the variable diameter can decrease from a starting diameter by radially inwardly deflection of the two or more elongated members toward the central axis, such as in response to engaging the sidewall 20 during insertion into the prosthesis receiving portion 14 (see, e.g., FIGS. 6 and 7).

In the example of FIG. 1, the pusher member 40 includes a second rod 50 that extends axially from a first end 52, which is proximal the first end 42, and terminates in a distal second end 54. The second rod 50 can be coaxial with the first rod 42, although it need not be coaxial (e.g., it might be transverse). The second rod 50 also has diameter 56 which may also be substantially fixed along its length, and which is different from the diameter 46 of the rod 42. The diameter 56 of the second rod 50 is also less than the diameter 22 of the opening 16, such that the rod 50 can be inserted axially, at least partially into the passage defined by the sidewall 20.

The pusher member 40 can also include a spacer 58 that extends radially outwardly from the pusher member at an axial location that is between the first and second rods 42 and 50, respectively. The spacer 58 thus separates the rods 42 and 50. The spacer 58 can also extend radially beyond the exterior of each of the rods to provide a diameter that is greater than the diameter 22 of the opening 16. By configuring the spacer 58 to be diametrically larger than the opening 16, it provides a convenient handle for grasping the pusher member 40. The spacer 58 can also engage the opening 16 of the guide member 12 to inhibit insertion of the pusher member beyond some predetermined distance.

By way of example, assuming that the rod 50 has a greater cross-sectional diameter than the rod 42, the larger diameter rod 50 can be used to urge the prosthesis into the prosthesis receiving portion 14 while the prosthesis itself has a greater diameter. After the prosthesis has been inserted a first amount using the second rod 50, the user can flip the pusher member (e.g., 180 degrees) so that the first, smaller diameter rod 42 is axially aligned with and adjacent to the prosthesis receiving portion 14. The user can employ the rod 42 to push the prosthesis further into the prosthesis receiving portion 14 and through the exit aperture 18 and into the lumen 26 (e.g., engaging the shoulder 34). By placing the barrel of the implanter within the lumen 26, the prosthesis can be conveniently loaded into the barrel.

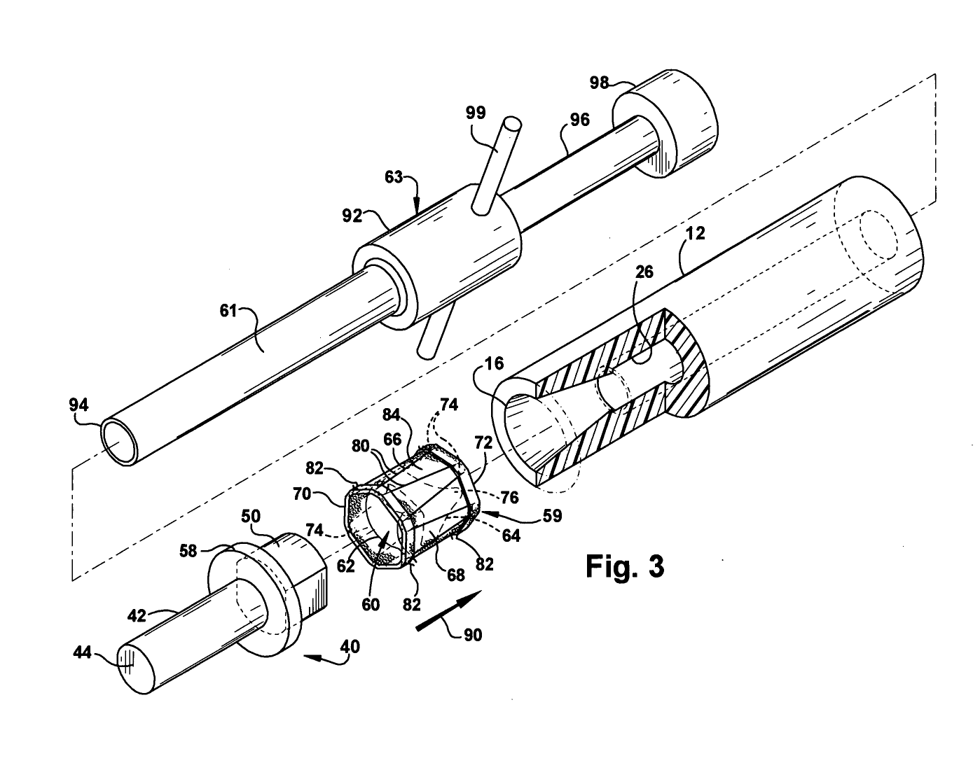

FIGS. 3, 4 and 5 depict different parts of a procedure that can be employed to load a prosthesis 59 into a barrel 61 of an implanter 63 according to an aspect of the present invention. For purposes of simplicity explanation (but not by way of limitation), the procedure is implemented using the loading system 10 shown and described with respect to FIGS. 1 and 2.

By way of further example, the procedure shown in FIGS. 3, 4 and 5 will described in the context of loading a natural tissue heart valve prosthesis 59 into the implanter 63. The natural tissue heart valve prosthesis 59 includes a valve 60 having an inflow end 62 and an outflow end 64 at axially opposed ends of the valve. The valve 60 is mounted within a support 66. For instance, a sidewall portion 68 of the valve 60 extends between the ends 62 and 64 of the valve, and between corresponding ends 70 and 72 of the support 66. That is, the inflow end 62 of the valve 60 is positioned near an inflow end 70 of the support 66 and the outflow end 64 of the valve is positioned near an outflow end 72 of the support. The outflow end 64 of the valve 60 can have a generally sinusoidal contour, as shown in FIG. 3, although the valve is not limited to such an outflow contour. For the example valve 60, the peaks of the sinusoidal outflow end 64 can be aligned generally with and attached to support junctures 74 near the end 72 of the support 66. The valve 60 can be connected within the support 66 via sutures or other known connecting means, for example.

The valve 60 is configured to provide for substantially unidirectional flow of blood through the valve. In the example of FIG. 3, the valve 60 includes a plurality of leaflets 76 that extend radially inward from the sidewall portion 68 of the valve. The leaflets 76 are moveable into and out of engagement with each other to coapt for providing unidirectional flow of blood through the valve 60. For different types of valves, there may be different numbers of leaflets or other moveable means (e.g., a ball, a flap or other structure) that provide for the desired unidirectional flow of blood through the valve. Additionally, the valve 60 can be a homograft or xenograft or, alternatively, the valve can be constructed of natural tissue, synthetic or a combination of natural and synthetic materials that are connected together to provide the valve. For example, the valve 60 can be similar to the type of valve shown and described in U.S. Pat. Nos. 5,935,163, 5861,028 or 5,855,602, as well as other types of valves mentioned herein as well as may otherwise be known or yet to be developed.

It is to be understood and appreciated that various types of valve configurations of could be employed to provide the prosthesis 59 in accordance with an aspect of the present invention. For example, the valve 60 can include one or more leaflets mounted within a length of tubular valve wall or other generally cylindrical biocompatible material and operate in a known manner to provide for the unidirectional flow of fluid through the valve from the inflow to outflow ends. By way of further example, when the prosthesis is to be implanted at the pulmonary position, the valve 60 can be a treated pulmonic valve (e.g., homograft or xenograft). When it is to be implanted at an aortic position, the valve 60 can be a treated aortic valve (e.g., homograft or xenograft). Alternatively, the valve 60 can be manufactured from natural tissue (e.g., animal pericardium, dura matter) and/or synthetic materials to provide for desired unidirectional flow of blood.

In the example of FIG. 3, the support 66 is configured to enable the valve to be compressed to a reduced cross-sectional dimension (diameter) and then expanded back to an expanded condition. The support 66 can be self-expanding from its reduced cross-sectional dimension or it may be expandable by employing other means to expand the valve manually (e.g., balloon catheter or other radially expanding mechanism). The support 66 includes substantially axially extending support features 80 that interconnect the respective support junctures 74. In the example of FIGS. 3, 4 and 5, the support junctures 74 are configured as arcuate junctures that are biased so as to urge a pair of adjacent support features 80 circumferentially apart. The arrangement of support junctures 74 and corresponding axially extending support features 80 can thus define a substantially sinusoidal sidewall portion of the support 66 having a substantially cylindrical configuration.

In the example of FIGS. 3, 4 and 5, there are six junctures at each of the respective ends 70 and 72 that are interconnected by associated support features 74, although a support is not limited to any particular number of features. Those skilled in the art will understand and appreciate that other numbers (e.g., 2, 3, 9, 12 and so forth) and different configurations of end junctures 74 can be utilized. For example, as an alternative to curved interconnecting end junctures 74 shown herein, such ends could be pointed or rectangular or include one or more windings at each juncture.

The support 66 further includes one or more projections or spikes 82 that extend axially and radially outwardly from at least some of the respective end junctures 74 of the support. While a pair of such spikes 82 is illustrated as associated with each end juncture 74, other number of spikes can be implemented, such as single spike or more than two spikes at some or all of the junctures. In the example illustrated in FIGS. 3, 4, and 5, the pairs of spikes at opposite ends operate to mitigate movement in different directions, such as by having each spike 82 forming an acute angle relative to its associated support feature 82 from which it extends.

According to one aspect of the present invention, the support can be formed a shape memory material, such as NITINOL. For example, the support can be formed from a small cylindrical tube of the shape memory material, such as via a laser cutting (ablation) process in which the desired sinusoidal sidewall is cut from the tube. In this way, the support features 80, the interconnecting end junctures 74, and associated spikes 82 can be formed as a monolithic structure (e.g., integrally formed) having a desired shape and size. Additionally, ends of the spikes 82 can have tapered or sharpened tips to facilitate gripping surrounding tissue when implanted. For example, the spikes 82 can be formed by laser cutting from the same tube or, alternatively, they could be welded onto or otherwise attached to the support 66 at desired positions. The resulting structure can then be heated to its transformation temperature and forced to a desired cross-sectional dimension and configuration (its austenitic form), such as shown in FIGS. 3, 4 and 5. The support 900 can then be bent or deformed to a reduced cross-sectional dimension when in its low-temperature (martensitic) form to facilitate its mounting within a barrel of a implanter, for example.

Those skilled in the art will appreciate various other materials that may be utilized for the support 66, including elastically deformable and inelastically deformable materials, such as metals, alloys and plastics or other polymers and combinations of materials. By elastically deformable, it is meant that the structure is capable of sustaining stress without permanent deformation, such that it tends to return substantially to its original shape or state when the applied stress is removed (e.g., self expanding from its reduced cross-section). By inelastically deformable, it is meant that the structure substantially retains its deformed shape after sustaining stress, such that it bends and stays bent until deformed to another (e.g., its original) shape or configuration. Additionally, if something is described herein as being deformable it may be either elastically deformable or inelastically deformable or exhibit different characteristics of one or both of such deformability.

The prosthesis 59 may also include an outer sheath 84 of a substantially biocompatible material. The outer sheath 84 covers at least a substantial amount of exposed portions of the support 66, such as including the ends 70 and 72, to mitigate contact between the blood and the support when the prosthesis is implanted. The valve 60 further can be attached relative to the sheath 84, such as by sutures along the inflow and outflow ends of the valve. Such sutures (not shown) further can connect the valve 60 and the sheath 84 relative to the support 66. The outer sheath 84 can cover the entire support 66, such that all non-biological material is completely covered, for example. The outer sheath 84 can be formed of one or more NO-REACT® natural tissue sheets (e.g., animal pericardium), although other natural materials (e.g., dura matter, collagen), synthetic biocompatible materials or combinations of natural and synthetic materials can also be used to provide a biocompatible outer sheath.

In the example of FIG. 3, the implanter 63 includes an elongated cylindrical barrel 61 that extends from a body portion 92 and terminates in an open end 94. The barrel 61 has an inner diameter that may vary according to the type of prosthesis 59 as well as the dimension and configuration of the prosthesis being implanted. The implanter 63 can include a plunger 96 that is configured for axial movement within the barrel 61. That is, the plunger 96 can be urged or activated for axial movement through the barrel 61 by employing a knob 98 that is operatively connected (directly or indirectly) with the plunger. For instance, a user can push the knob 98 with the user's thumb while holding a handle or flange 99 with the user's index finger and middle finger (e.g., similar to using a syringe). Other means (e.g., trigger, spring activated, threads, etc.) can be employed for moving the plunger 98 in a desired direction. Additionally, after discharging the prosthesis 59 from the barrel 61, the plunger 96 may be removed so that the barrel provides a passage through which a corresponding implantation site (near the end 94) and the implanted prosthesis can be accessed.

By way of further example, the loading procedure can begin by selecting the appropriate prosthesis, which in this example is an expandable type natural tissue heart valve prosthesis 59 described above. As described herein, however, the loading system 10 is not limited to use with such a heart valve prosthesis. For the example heart valve prosthesis 59, the valve 60 is axially aligned with the opening 16 of the prosthesis receiving portion 14 with the inflow end 62 and the outflow end 64 axially arranged according to where the valve is to be implanted and the direction from which the implanter is going to be positioned. Thus, in the illustrated example of FIG. 3 (and not by way of limitation), the prosthesis 59 is being inserted into the guide 12 with the valve outflow end 64 of the valve 60 adjacent the opening. The initial alignment and insertion of the prosthesis 59 into the prosthesis receiving portion 14 can be implemented manually (e.g., by hand).

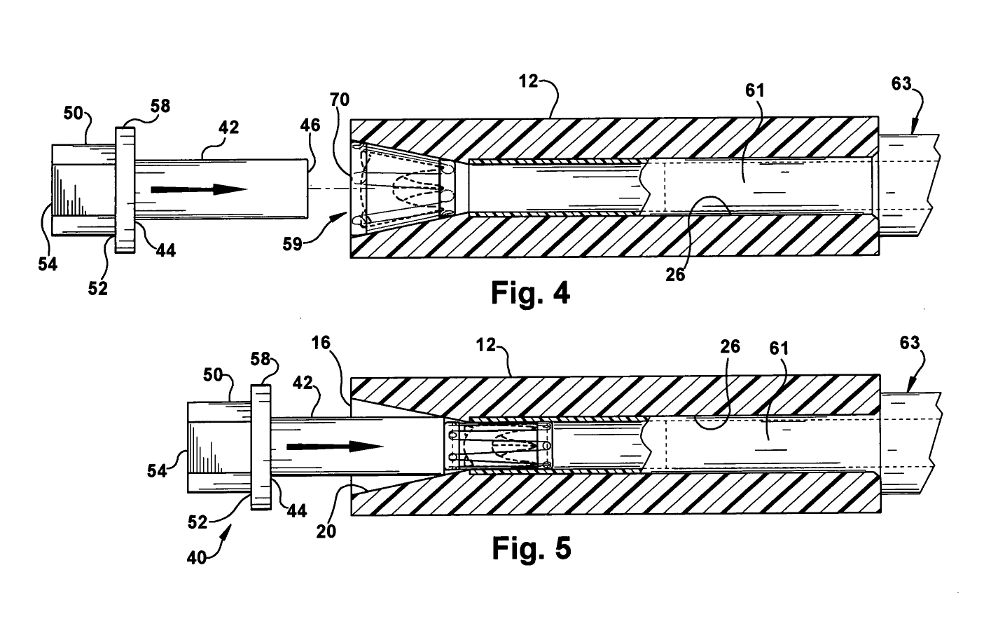

Once the prosthesis 59 has been appropriately aligned and, optionally, inserted into the opening a small amount (e.g., about 2-5 mm), the pusher member 40 can be employed to urge the prosthesis 59 farther into the guide member 12. For example, the larger diameter rod 50 can be employed first to urge the prosthesis 59 into the guide by causing the surface at the end 54 to contact the end 70 of the support 66. The pusher member 40 can urge the prosthesis in the direction of arrow 90 axially into the passage provided by the prosthesis receiving portion 14 of the guide member 12. The engagement between the sidewall 20 of the prosthesis receiving portion 14 and the exterior of the prosthesis 59 as the prosthesis is urged axially into the guide member 12 compresses the prosthesis 59 to a reduced cross sectional dimension, as shown in FIG. 4. For instance, the inflow end 70 of the prosthesis remains in a substantially expanded condition, whereas portion of the prosthesis sidewall proximal the outflow end (located within the sidewall of the prosthesis receiving portion 14) tapers along its length according to the dimensions and configuration of the sidewall 20 in which it is being inserted.

After the rod 50 has been inserted into the prosthesis receiving portion 14 such that it cannot be inserted further (e.g., the end 54 engages the sidewall 20 or the central spacer 58 engages the rim at the opening 16), the pusher member 40 can be flipped around to use the smaller diameter rod 42. For example, in FIG. 4, the rod 42 is axially aligned with the prosthesis 59 and the guide member 12. The end 44 of the pusher member 40 can, in turn, be urged into engagement with the adjacent end 70 of the prosthesis 59 so as to insert the prosthesis into the guide member for loading the prosthesis farther into the barrel 61 of the implanter 63, such as shown in FIG. 5.

The rod 40 (having a smaller diameter than the rod 50) thus can be inserted axially into the prosthesis receiving portion 14 of the guide member 12 further than the rod 50. The distance that the rod 40 can be inserted will generally depend on the relative diameters of the rod and the sidewall 20. In the example of FIG. 5, the rod 40 is inserted approximately 3/4 the length of the sidewall 20 of the tissue receiving portion when the end 44 engages the sidewall so as to inhibit further movement into the guide member 12. It will be appreciated that the rod 40 and guide member could be provided at different relative dimensions from those shown so as to permit different depths of insertion. Additionally, more than two rods can be provided to allow for additional levels axial insertion. For smaller size barrels (having a diameter from about 7 mm to about 9 mm), the pusher can include one or more rods configured to have a variable diameter so that the pusher member 40 can be inserted axially at or adjacent to the exit aperture 18 of the prosthesis receiving portion 14.

As shown in FIGS. 6, 6A, 7 and 7A, a pusher member 100 includes at least one elongated rod assembly 102 having two elongated rod members 104 and 106. The guide member 12 can be the same or different from that shown and described with respect to FIGS. 1-5; although, for sake of simplicity of explanation, will be described as being the same guide member 12, as shown and described herein. The rod assembly 102 is not limited to only two rod members 104 and 106, as more than two rod members can be implemented (e.g., a substantially circumferential array of three, four or other numbers of axially extending rod members spaced apart from each other). Each of the rod members 104 and 106 are joined at and extend axially from a first end 108 and terminate to define respective second ends 110 and 112 of the pusher member. The second ends 110 and 112 of the rod members 104 and 106 are substantially flat (e.g., substantially coplanar) or otherwise configured for engaging an adjacent end of the prosthesis 59. In the example of FIG. 6, the rod members 104 and 106 are coextensive and substantially parallel and spaced apart from each other by slot or notch 114 that extends continuously and axially from the end 108 to the open end between the ends 110 and 112. The first end 108 can operate as a hinge that permits the ends 110 and 112 of the rod members 104 and 106 to deflect radially inwardly relative to the central axis (and toward each other) to reduce the distance between the opposing side surfaces of the respective rods. Additionally, diametrically opposed side edges 113 and 115 of the respective rod members 104 and 106 can be spaced apart from each other a distance that approximates the diameter (e.g., reference number 24 in FIG. 2) of the exit aperture 18. While in the example of FIGS. 6 and 7 the rod assembly 102 is depicted as an integral structure (e.g., monolithic), the rod members 104 and 106 could be fixed relative to each other by one or more other structures (e.g., hinge, spring, rivot, etc.) that permits desired movement (e.g., radially deflection) of the rod members to a reduced cross-section.

For example, radial thickness of each of the rod members 104 and 106 at the ends 110 and 112, respectively, can be dimensioned so that when the rod members deflect toward and into engagement with each other, the total reduced thickness approximates the diameter (e.g., reference number 24 in FIG. 2) of the exit aperture 18. In this way, the variable diameter of the rod assembly 102 can decrease from a starting diameter (FIG. 6) and decrease radially due to inward deflection of the elongated rod members 102 and 104 toward each other. The deflection of the rod members 104 and 106 toward each other thus results in opposing inner surfaces 117 and 119 moving from a spaced apart condition (FIG. 6A) to a second condition in which the opposing surfaces are closer or contacting each other (FIG. 7A). Such inward deflection can occur in response to the exterior surface of the rod members 102 and 104 engaging the sidewall 20 during insertion into the prosthesis receiving portion 14 (FIG. 7). Alternatively, the inward deflection of the elongated rod members may be manually adjustable, such as by application of external force or by otherwise adjusting the distance between the surfaces 117 and 119.

The pusher member 100 can include another rod 120 that extends axially from a spacer 122, which is located intermediate the rod 120 and the variable rod assembly 100. The rod 120 extends from the spacer and terminates in a second end 124. The rod 120 can be coaxial with the first rod assembly 100, although it need not be coaxial (e.g., it might be transverse or at other relative angular orientations). In the example, of FIGS. 6 and 7, the rod 120 also has diameter which may be substantially fixed along its length, which can be larger than the starting diameter of the rod assembly 100. Alternatively, the rod 120 can be configured to have a variable diameter similar to the rod assembly 100, but have different starting and ending diameters. In this way, the rod 120 can be used for an initial phase of inserting the prosthesis 59 into the prosthesis receiving portion (e.g., similar to as shown and described in FIG. 3). The variable rod assembly 100 can be used to complete the insertion of the prosthesis into and through the prosthesis receiving portion 14 and loading into the barrel of the implanter, such as depicted in FIG. 7.

It will be understood that the loading system and procedure are not limited to use with a particular type of heart valve prosthesis 59. Other types of cardiovascular prostheses, which are deformable to a reduced diameter and expandable to an expanded condition, can also be used. As described herein, for example, the prosthesis could be a stent (e.g., for a heart valve or for a blood vessel), a venous valve, a mechanical heart valve, a biomechanical heart valve, or a different type of natural tissue heart valve from that shown herein.

What has been described above includes examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications and variations that fall within the spirit and scope of the appended claims.

What is claimed is:

1. A system for loading an implantable prosthesis into an implanter, comprising:
a prosthesis receiving portion having an opening at a first end that is spaced axially apart from a second end by a sidewall portion, the sidewall portion having a substantially smooth, radially inner sidewall that tapers from the from a first diameter at the opening to a second diameter at the second end of the prosthesis receiving portion, the second diameter being less than the first diameter and being greater than the prosthesis and defining an exit aperture of the prosthesis receiving portion;
a receptacle extending from the second end of the prosthesis receiving portion, the receptacle being dimensioned and configured for coaxially receiving a portion of the implanter at a location relative to the exit aperture that provides a fluid transition between from the exit aperture of the prosthesis receiving portion to the receptacle, whereby loading the prosthesis from the within the prosthesis receiving portion into the hollow portion of the implanter is facilitated.

2. The apparatus of claim 1, wherein the receptacle further comprises an elongated body having an aperture that extends longitudinally through the elongated body from the second end of the prosthesis receiving portion substantially coaxial with the exit aperture and terminates in an opening at a proximal end thereof that is axially spaced apart from the second end of the prosthesis receiving portion.

3. The apparatus of claim 2, wherein the aperture extending through the elongated body has a diameter that at least approximates the second diameter.

4. The apparatus of claim 3, wherein the axial length of the receptacle is greater than the axial length of the prosthesis receiving portion.

5. The apparatus of claim 2, further comprising a continuous exterior sidewall that extends around the prosthesis receiving portion and the receptacle between first end of the prosthesis receiving portion and the proximal end of the receptacle.

6. The apparatus of claim 2, wherein the diameter of the aperture extending through the elongated body is substantially fixed along the length of the elongated body.

7. The apparatus of claim 2, wherein the receptacle and the prosthesis receiving portion comprise a monolithic structure that defines a guide member.

8. The apparatus of claim 1, further comprising an elongated pusher member having at least one rod that has an exterior sidewall that extends from a first end and terminates in a second end spaced longitudinally apart from the first end, the exterior sidewall having an outer diameter proximal the second end thereof that is between the first diameter and the second diameter.

9. The apparatus of claim 8, wherein the at least one rod further comprises at least two rod members that extend substantially parallel to each other in a spaced apart relationship, the at least two rods being fixed relative to each other at the first end and being coextensive along a length thereof from the first end to the respective second ends thereof, each of the at least two rod members being inwardly deflectable relative to a longitudinal central axis of the pusher member so as to vary the space between the at least two rods near the respective second ends thereof.

10. The apparatus of claim 6, wherein the at least rod is a first rod, the pusher member further comprises a second elongated rod that is spaced axially apart from the at least one rod by a spacer, the second elongated rod having an exterior sidewall that extends from the spacer and terminates in a second end thereof that is spaced longitudinally apart from the spacer, the spacer having a diameter that is at least the diameter of the second elongated rod and that is greater than the outer diameter of the firs rod.

11. The apparatus of claim 1, wherein the receptacle further comprises an elongated sidewall having a aperture extending therethrough that is dimensioned and configured for receiving at least a portion of an elongated barrel of the implanter therein and for aligning an opening of the barrel substantially coaxial with the exit aperture of the prosthesis receiving portion.

12. The apparatus of claim 9, wherein the receptacle has a diameter that is greater than the diameter of the exit aperture, such that the juncture from the receptacle aperture to exit aperture of the prosthesis receiving portion defines a shoulder that inhibits insertion of the barrel axially beyond the shoulder while providing for fluid communication from the interior of the prosthesis receiving portion into the receptacle aperture.

13. A system for preparing a prosthesis for in vivo implantation, the system comprising:
a guide member comprising:
a prosthesis receiving portion having a substantially smooth, radially inner sidewall having a conical frustum cross sectional configuration that tapers from the from a first diameter at an opening at a first end of the guide member to a smaller second diameter at the second location within the guide member that is spaced apart from the first end; and
a receptacle located adjacent the second end of the sidewall portion; and
an implanter having an elongated barrel having a lumen configured for receiving the prosthesis in a reduced cross-sectional dimension, the receptacle being dimensioned and configured for aligning an opening of the barrel with the second location within the guide member, whereby loading the prosthesis from the within the guide into the portion of the implanter is facilitated.

14. The system of claim 13, further comprising an elongated pusher member having at least one rod having an exterior sidewall that extends from a first end and terminates in a second end spaced longitudinally apart from the first end, the exterior sidewall having an outer diameter that is substantially between the first diameter and the second diameter.

15. The system of claim 14, wherein the at least one rod further comprises at least two rod members that extend substantially parallel to each other in a spaced apart relationship, the at least two rods being fixed relative to each other at the first end and being coextensive along a length thereof from the first end to respective second ends thereof, each of the at least two rod members being inwardly deflectable relative to a longitudinal central axis of the pusher member so as to vary the space between the at least two rods near the respective second ends thereof

16. The system of claim 13, wherein the receptacle further comprises an elongated body having an aperture that extends longitudinally through the body from the second end of the guide portion substantially coaxial with the inner sidewall of the prosthesis receiving portion and that terminates in an opening at a proximal end thereof that is axially spaced apart from the second end of the elongated body.

17. The system of claim 16, wherein the aperture extending through the elongated body has a diameter that at least approximates the second diameter to provide for fluid communication from within the prosthesis receiving portion into the receptacle aperture.

18. The system of claim 17, wherein the axial length of the receptacle aperture is greater than the axial length of the prosthesis receiving portion, and the diameter of the receptacle aperture extending through the elongated body is substantially fixed along the length of the elongated body.

19. A method of using the system of claim 13, the method comprising:
inserting the barrel of the implanter within the aperture so that the opening of the barrel is adjacent and aligned with the exit aperture of the prosthesis receiving portion;
positioning a deformable prosthesis at the opening of the prosthesis receiving portion;
urging the prosthesis axially into the prosthesis receiving portion such that the inner sidewall of the prosthesis receiving portion engages the exterior of the prosthesis and causes a cross-sectional dimension of the prosthesis to reduce commensurate with the cross sectional dimension of the inner sidewall being engaged by the prosthesis;
pushing the prosthesis through the exit aperture and through the opening of the barrel such that at least a portion of the prosthesis resides within the barrel of the implanter.

20. The method of claim 19, wherein at least one of the urging and the pushing is performed using a pusher member, the pusher member comprising at least one elongated rod having an exterior sidewall that extends from a first end and terminates in a second end spaced longitudinally apart from the first end, the exterior sidewall having an outer diameter that is substantially between the first diameter and the second diameter.

21. The method of claim 19, wherein the prosthesis comprises a heart valve prosthesis.
